(19) United States
(12) Patent Application Publication
(10) Pub. No.: US 2022/0354386 A1
(43) Pub. Date: Nov. 10, 2022

(54) APPARATUS, METHOD AND ASSOCIATED SENSOR HEAD FOR SHIVERING DETECTION

(71) Applicant: BrainCool AB, Lund (SE)

(72) Inventor: David MCGINNIS, Lund (SE)

(73) Assignee: BrainCool AB, Lund (SE)

(21) Appl. No.: 17/637,016

(22) PCT Filed: Aug. 19, 2020
(86) PCT No.: PCT/EP2020/073267

§ 371 Date: Feb. 21, 2022

(30) Foreign Application Priority Data
Aug. 23, 2019 (SE) 1950967-8

(51) Int. Cl. A61B 5/11; A61F 7/00

(52) U.S. Cl. CPC A61B 5/1101; A61F 7/0085; A61F 2007/0056

(57) ABSTRACT

A shivering detection apparatus (100) for detecting shivering of a patient (150) is provided. The apparatus (100) comprises a control unit (110) operatively connected to a transmit unit (120), for transmitting a transmit signal (170) based on a reference signal (140) of a transmit frequency towards the patient (150). The control unit is further operatively connected to a receive unit (130), for receiving a receive signal (190) as a portion of a reflected signal (180) comprising a portion of the transmit signal (170) reflected by the patient (150). The control unit (110) is configured to compare the receive signal (190) to the reference signal (140) and to detect shivering of the patient (150) as differences between the receive signal (190) and the reference signal (140). An associated method and sensor head is also provided.

### TECHNICAL FIELD

The present invention relates to detection of shivering of a person and more precisely to the detection of shivering of a person undergoing therapeutic hypothermia.

### BACKGROUND

Medical cooling of parts of the human body is a useful tool to reduce adverse impacts on the human body under certain conditions. For example, medical cooling may be used for cooling the brain of patient suffering from circulatory emergencies e.g. a stroke or cardiac arrest. By reducing the temperature of the brain, the risk of tissue damage is reduced for those parts of the brain lacking an adequate oxygen supply during the circulatory emergency.

A person suffering from e.g. cardiac arrest may be subject to non-invasive medical cooling, therapeutic hypothermia. A general effect of such treatment is that the body of the patient will start to shiver as a result of the cooling. Under normal conditions when the body temperature decreases below a certain point, e.g. around 35° C., the body starts to shiver in an attempt of regaining the heat loss. The involuntary shivering may be specific to each person undergoing therapeutic hypothermia and may consequently occur at different temperatures. Shivering is undesirable since it consumes the patient's energy. Moreover, under prolonged cooling treatment, the patient may also have a fever and in that case the cooling treatment may result in even higher body temperatures.

One problem with therapeutic hypothermia is that shivering must be detected or avoided. To avoid shivering of a patient, cooling systems may be provided with heating circuits to control the temperature of the cooling fluid or part of the patient that are not undergoing therapeutic hypothermia. To date, the most efficient and reliable shivering detection is performed by manual monitoring of the patient by trained staff. This is a resource consuming, tedious and error prone method of detection and there are some attempts at automating the detection.

Electromyography (EMG) is an electrodiagnostic medicine technique for evaluating and recording the electrical activity produced by skeletal muscles. In U.S. Pat. No. 8,706,207, a method and apparatus for treating shivering during therapeutic temperature control is presented. For detection of shivering, electrode-based detection using two or more electrodes arrayed to obtain the surface EMG data is described.

EMG recording systems are stationary, bulky, complex, expensive and sensitive to noise. From the above it is understood that there is room for improvements.

### SUMMARY

An object of the present invention is to provide a new type of shivering sensing apparatus which is improved over prior art and which eliminates or at least mitigates the drawbacks discussed above. More specifically, an object of the invention is to provide a shivering detecting apparatus that is cheap, simple, accurate and efficient. These objects are achieved by the technique set forth in the appended independent claims with preferred embodiments defined in the dependent claims related thereto.

In a first aspect, a shivering detection apparatus for detecting shivering of a patient is presented. The apparatus comprises a control unit that is operatively connected to a transmit unit. This is for transmitting a transmit signal based on a reference signal of a transmit frequency towards the patient. The apparatus further comprises a receive unit. This is for receiving a receive signal as a portion of a reflected signal comprising a portion of the transmit signal reflected by the patient. The controlling unit is configured compare the receive signal to the reference signal and to detect shivering of the patient as differences between the receive signal and the reference signal.

In a first variant of the apparatus, the control unit comprises a multiplying unit suitable for multiplying the receive signal with the reference signal. This is done in order to provide one or more down converted receive signals centered at 0 Hz. The apparatus further comprises a filtering unit for low pass filtering the down converted receive signals to provide one or more receive channels, and a converting unit for converting the said one or more receive channels to provide one or more digital receive channels. Further to this, the apparatus comprises a processing unit for detecting shivering of the patient based on the one or more digital receive channels. By employing this coherent detection where the receive signal is compared to the same reference signal as the transmit signal is based on, the total distance from the transmit unit to the receive unit via reflection off the patient, is isolated from any shivering of the patient.

In a further variant, the apparatus the multiplying unit comprises a first multiplier arranged to multiply the receive signal with an in phase reference of the reference signal to provide an in phase receive channel, and a second multiplier arranged to multiply the receive signal with a quadrature phase reference of the reference signal to provide a quadrature phase receive channel. The quadrature phase reference is of an orthogonal phase to the in phase reference. By having an in phase channel and a quadrature phase channel common mode effects can be substantially eliminated, the phase shift due to the distance between the apparatus and the patient will manifest itself as a common mode effect.

In yet another variant of the apparatus, the converting unit comprises a first analogue to digital converter which is arranged to convert the in phase receive channel in order to provide a digital in phase receive channel. Also, the converting unit comprises a second analogue to digital converter arranged to convert the quadrature phase receive channel to provide a digital quadrature phase receive channel. One effect of digitalizing the channel(s) is that it enables advanced signal processing or alternatively, cheap detection using off the shelf cheap processing units. This will reduce the cost of the apparatus even further.

In another variant of the apparatus, the processing unit is configured to add the digital in phase receive channel and the digital quadrature phase receive channel to provide a complex sum, and to subject the complex sum to a Fast Fourier Transform, FFT, to provide a frequency representation of the complex sum. The complex sum will provide a shivering vector whose angle and amplitude will vary with the frequency of the shivering. This frequency will be clearly visible in the frequency representation provided by the FFT.

In an even further variant of the apparatus, shivering is identified if the frequency representation of the complex sum comprises at least one frequency component above a shivering threshold. One effect of this is that a shivering threshold can be defined and tuned to specific conditions.

In one variant, the apparatus further comprises the providing of a shivering control signal for controlling apparatuses external to the shivering detection apparatus and/or for alerting personnel to the presence of shivering. One effect of this is that the apparatus may be integrated into other medical equipment and/or used as a stand alone solution for alerting personnel to shivering without having dedicated resources monitoring the patient.

In another variant of the apparatus, the transmitter is an ultrasonic transducer for transmitting an acoustic signal and the receiver is an ultrasonic receiver for receiving an ultrasonic acoustic signal. By using ultrasound and acoustic waves, the risks related to e.g. electromagnetic radiation are avoided. Electromagnetic energy will, in part, be absorbed in the patient and may cause heating and consequently affect the shivering. The comparably low frequency of the ultrasound will enable cheaper processing of the receive signal by e.g. off the shelf component and the total cost of the apparatus is reduced.

In an even further variant of the apparatus, said one or more multipliers is a logical exclusive-or, XOR, gate. Implementing the multiplier as one or more XOR-gates in an extremely cost effective way of down converting the receive signal thus reducing the cost of the apparatus further.

In yet another variant of the apparatus, the receive signal and the reference signal are normalized to a common level prior being provided to the multiplying unit. This removes any problems relating to the receive signal being of lower amplitude than the reference signal, such amplitude differences may occur due to e.g. attenuation of the transmit and reflected signals.

In another variant of the apparatus, the normalizing is realized by one or more analogue comparators. A comparator is a very cheap off the shelf component that further reduces the cost of the apparatus. Also, one effect of the comparator is that it has a squaring effect on the signal being normalized which further simplifies the logical processing.

In a last variant of the patient is subjected to therapeutic hypothermia. One effect of this is that the effect of the therapeutic hypothermia can be closely monitored and patient safety can be maximized.

In a second variant, a shivering detection method for detecting shivering of a patient is presented. The method comprises transmitting (1210), wirelessly by a transmit unit (120), a transmit signal (170) of a transmit frequency (fₜₓ) based on a reference signal (140) towards the patient. It also comprises receiving, by a receive unit, a reflected signal as a portion of the transmit signal reflected by the patient, to provide a receive signal. Further to this, the method comprises multiplying, by one or more multipliers, the receive signal with the reference signal to provide one or more down converted receive signals centered at 0 Hz. It also comprises filtering, by a low pass filter, said one or more down converted receive signals to provide one or more receive channels and converting, by at least one analogue to digital converter, said one or more receive channels to provide one or more digital receive channels. The one or more digital receive channels is used to detecting, by a processing unit, shivering of the patient.

In one variant of the method, the step of multiplying further comprises multiplying, by a first multiplier, the receive signal with an in phase reference of the reference signal to provide an in phase receive channel, and multiplying, by a second multiplier, the receive signal with a quadrature phase reference of the reference signal to provide a quadrature phase receive channel. The quadrature phase reference is of an orthogonal phase to the in phase reference. By having an in phase channel and a quadrature phase channel common mode effects can be substantially eliminated, the phase shift due to the distance between the apparatus and the patient will manifest itself as a common mode effect.

In another variant of the method, the step of converting further comprises converting, by a first analogue to digital converter, the in phase receive channel to provide a digital in phase receive channel, and converting, by a second analogue to digital converter, the quadrature phase receive channel to provide a digital quadrature phase receive channel. One effect of digitalizing the channel(s) is that it enables advanced signal processing or alternatively, cheap detection using off the shelf cheap processing units. This will reduce the cost of the apparatus even further.

In a further variant of the method, the step of detecting further comprises, by the processing unit, adding the digital in phase receive channel and the digital quadrature phase receive channel to provide a complex sum, and subjecting the complex sum to a Fast Fourier Transform, FFT, to provide a frequency representation of the complex sum. The complex sum will provide a shivering vector whose angle and amplitude will vary with the frequency of the shivering. This frequency will be clearly visible in the frequency representation provided by the FFT.

In a third aspect, a sensor head for use with the shivering detection apparatus for detecting shivering of a patient is presented. The sensor head comprises the transmit unit and the receive unit of the shivering detection apparatus. The transmit unit and the receive unit are arranged in the sensor head such that a transmit signal of a transmit frequency transmitted by the transmit unit is reflected by the patient at an incident angle forming a reflected signal and at least a portion of the reflected signal is detectable by the receive unit. One effect of the sensor head is that it enables the transmit unit and receive unit can be arranged in, relative to one another, fixed positions allowing the total distance from the transmit unit to the receive unit via reflection off the patient can be controlled.

In one variant of the sensor head, the incident angle is less than 55 degrees. One effect of this is that the amount of the transmit signal that is reflected of the patient and receivable by the receive unit is greatly decreased if the angle is above 55 degrees.

In a another variant of the sensor head, at least one inner surface of the sensor head is provided with a lining material that comprises absorbing features of signals of the transmit frequency. The lining material reduces unwanted reflections of the transmit signal of the inner surface of the sensor head. The unwanted reflections would be received as noise by the receive unit and the signal to noise ratio of the receive signal would decrease.

In yet another variant of the sensor head, the transmit signal is an ultrasonic acoustic signal, the transmit frequency is 40 kHz and the lining material is cotton cellulose cloth. By using ultrasound and acoustic waves, the risks related to e.g. electromagnetic radiation are avoided. Electromagnetic energy will, in part, be absorbed in the patient and may cause heating and consequently affect the shivering. The comparably low frequency of the ultrasound will enable cheaper processing of the receive signal by e.g. off the shelf component and the total cost of the apparatus is reduced. The cotton cellulose cloth has the effect of being particularly good at absorbing the ultrasonic acoustic transmit signal and consequently reducing unwanted reflections.

In a further variant of the sensor head, it further comprises at least two mounting tabs arranged to receive an elastic band and/or a removable collar clip. The mounting tabs enables the same sensor head to be used with different types of fastening means. This allows for a reduced number of sales items and storage space is reduced. Also, the flexibility in fastening means makes it easier to use in diverse situations.

In one variant of the sensor head, it is configured to be arranged on the temple of a patient by means of the elastic band and/or on the neck of the patient by means of the removable collar clips. The neck and the temple are parts of the patient that are prone to shivering and having the sensor head located at these places allows for accurate and early detection of shivering.

### BRIEF DESCRIPTION OF THE DRAWINGS

Embodiments of the invention will be described in the following; references being made to the appended diagrammatical drawings which illustrate non-limiting examples of how the inventive concept can be reduced into practice.

FIG. 1 is a schematic view of a shivering detection apparatus.

FIGS. 2a-b are schematic views illustrating Doppler shift of a reflected signal.

FIG. 3 is a schematic overview of one embodiment of a the shivering detection apparatus.

FIGS. 4a-c are diagrams showing the frequency content and frequency shift of the reflected signal.

FIG. 5 is a schematic overview of one embodiment of a the shivering detection apparatus.

FIGS. 6a-6d are diagrams depicting signals at different stages in the shivering detection.

FIG. 7 is a diagram showing in phase and quadrature phase signals when the received signal is phase shifted 180°.

FIG. 8 is a schematic view of a sensor head.

FIG. 9 is a schematic view of an incident angle.

FIG. 10 is an three dimensional view of one embodiment of a sensor head.

FIGS. 11a-b are schematic views of one embodiment of the sensor head with one embodiment of a fastening means.

FIG. 12 is a flow chart of one embodiment of a method for detecting shivering.

### DETAILED DESCRIPTION OF EMBODIMENTS

Hereinafter, certain embodiments will be described more fully with reference to the accompanying drawings. The invention may, however, be embodied in many different forms and should not be construed as limited to the embodiments set forth herein; rather, these embodiments are provided by way of example so that this disclosure will be thorough and complete, and will fully convey the scope of the invention, such as it is defined in the appended claims, to those skilled in the art.

In the following embodiments, examples and technical details, an apparatus, a method and an associated sensor head will be disclosed. The teachings are provided on a general level and the skilled person will realize that the embodiments given are but examples of many different ways of implementing the teachings.

The novel and ground breaking shivering detection method and apparatus of this disclosure is based on the insightful and inventive epiphany of the inventors that teaches that a signal reflected at a shivering body will be modulated by the frequency of the shivering. This allows for the design of a low cost and accurate electronic device that can be configured to provide a signal indicative of shivering of a patient. The device may be connected to other equipment e.g. equipment arranged to control the temperature of the shivering body. References throughout this text will be made to different types of shivering objects, e.g. shivering body, reflecting surface, patient, shivering element etc. The skilled person will realize that all these references will, in many examples, be equivalent or connected; the patient may for instance be partly fitted with a reflecting surface.

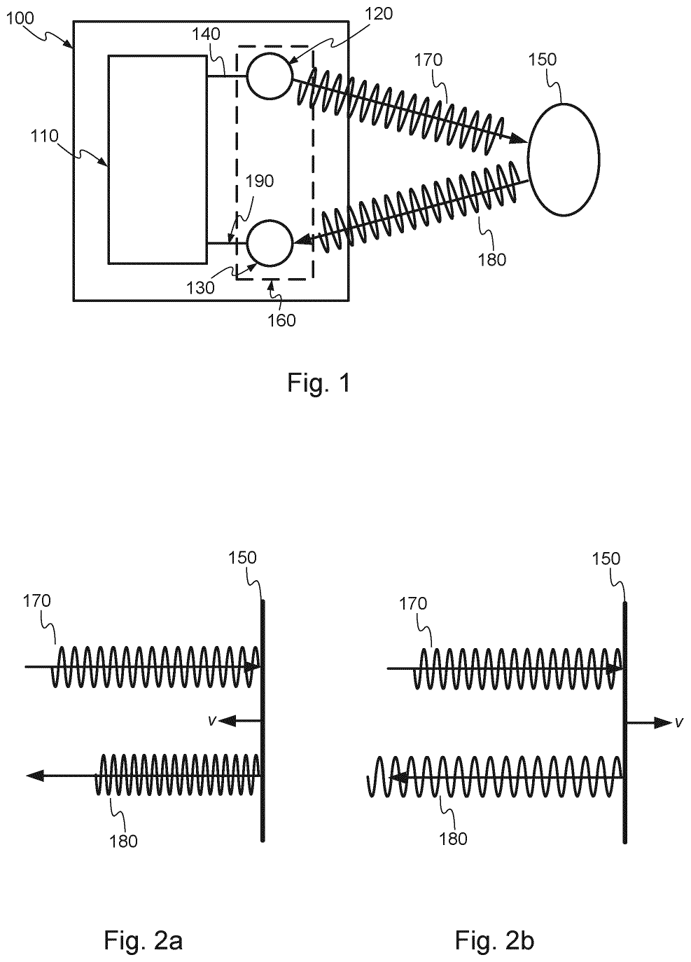

With reference to FIG. 1, an overview of a shivering detection apparatus 100 will be described. The shivering detection apparatus 100 comprises a controlling unit 110, a transmit unit 120, a receive unit 130. The transmit unit 120 is arranged to transmit a transmit signal 170 of a transmit frequency fₜₓ towards a reflecting surface 150. The transmit unit 120 receives a reference signal 140 of the transmit frequency fₜₓ activating the transmit unit 120. The reflecting surface 150 reflects at least a portion of the transmit signal 170 in a reflected signal 180 and the receiving unit 130 is arranged to receive at least a portion of the reflected signal 180 as a receive signal 190. The controlling unit 110 is configured to analyze the receive signal 190 by comparing it to the transmit signal 170 and determine if the reflecting surface 150 is shivering. Any shivering of the reflecting surface will, as will be detailed in the coming sections, manifest itself as frequency modulations to the transmit frequency fₜₓ. The transmit unit 120 and the receive unit 130 may be arranged in a sensor head 160.

The reflecting surface 150 may be any surface that is able to reflect a sufficiently large amount of the transmit signal 170 such that the reflected signal 180 is large enough to be received by the receiving unit 130 enabling the receiving unit 130 to provide a receive signal 190 to the controlling unit 110. The reflecting surface 150 may be different depending on the type of transmit signal 170 and depending on the transmit frequency fₜₓ of the transmit signal 170. If the transmit signal 170 is an audio signal and the transmit frequency fₜₓ is above the human hearing threshold, i.e. an ultrasonic acoustic transmit signal 170, the reflecting surface 150 may be e.g, the skin of a patient. Such an arrangement is especially beneficial since risks associated with electromagnetic radiation are removed. Such risks are heating of reflecting surface 150 affecting the shivering (some of the electromagnetic energy will typically be absorbed by the reflecting surface), burning of the reflecting surface 150 etc. If the transmit signal 170 is an electromagnetic signal and the transmit frequency fₜₓ is about 560 THz, i.e. a green laser transmit signal 170, the reflecting surface 150 may be e.g, the skin of a patient fitted with piece of reflecting material such as copper tape or similar. Naturally, the same reasoning applies to electromagnetic signals of lower or higher frequencies.

Looking to FIGS. 2a and 2b will help understand the basic concept of why the reflected signal 180 is modulated with the shivering of the reflected surface 150. The description below is given in relation to Doppler shifts at speeds significantly below the speed of light. The effects are the same for e.g. electromagnetic waves travelling at speeds in the range of the speed of lights, but the effect is called relativistic Doppler and the theory is somewhat more complex—but still known. In FIG. 2a, the reflected surface 150 is depicted as moving towards the source of the transmit signal 170 with a velocity v. This causes the wavelength of the reflected signal 180 to be reduced and consequently the frequency of the reflected signal will increase compared to the transmit frequency fₜₓ. In FIG. 2b, the reflecting surface is moving away from the source of the transmit signal 170 with a velocity v. In this example, the reflected signal 180 will have an increased wavelength compared to the transmit signal fₜₓ and consequently a lower frequency. The concept can be described by the Doppler effect and the transmit frequency fₜₓ will changed into a reflected frequency fᵣₓ as shown in Eqn. 1a below.

$${f}_{rx} = ( \frac{{v}_{0} \pm {v}_{r}}{{v}_{0} \pm {v}_{s}} ) \cdot {f}_{tx} \qquad Eqn . la$$

In Eqn. 1a, v₀ is the velocity of the signal in a medium where it travels, vᵣ is the velocity of a receiver relative to the medium and vₛ is the velocity of the transmitter relative to the medium. If the medium is vacuum and the signal is an electromagnetic signal, v₀ is the speed of light c. If the signal is an ultrasonic acoustic signal and the medium is air, v₀ is about 343 m/s. The velocity of the transmitter vₛ is positive if the source is moving away from the receiver and negative if the source is moving towards the receiver, the opposite is true for the velocity of the receiver vᵣ. This means that Eqn. 1b is true if the reflecting surface moves away from the source of the transmit signal 170 and Eqn. 1c holds if the reflecting surface moves towards the transmit signal. The locations of the source of the transmit signal 170 and the reception of the reflected signal 180 are assumed to be the same.

$${f}_{rx} = ( \frac{{v}_{0} - v}{{v}_{0} + v} ) \cdot {f}_{tx} \qquad Eqn . lb$$

$${f}_{rx} = ( \frac{{v}_{0} + v}{{v}_{0} - v} ) \cdot {f}_{tx} \qquad Eqn . lc$$

When the reflected surface 150 is shivering with a shivering frequency fₛ, the shivering will manifest itself as a change in velocity v of the reflecting surface 150 over time t and can be described according to Eqn. 2 below.

v(t)=vₛ cos(2πfₛt+θ) Eqn. 2

In Eqn. 2, vₛ is the maximum velocity of the shivering and θₛ is the phase of the shivering. Combining Eqns. 1b, 1c and 2 yields Eqn. 3 describing a frequency fᵣₓ of the reflected signal 180 as a function of time:

$${f}_{rx} ( t ) = ( \frac{{v}_{0} + {v}_{s} cos ( 2 π {f}_{s} t + {θ}_{s} )}{{v}_{0} - {v}_{s} cos ( 2 π {f}_{s} t + {θ}_{s} )} ) \cdot {f}_{tx} \qquad Eqn . 3$$

As seen in Eqn. 3, the the frequency fᵣₓ of the reflected signal 180 will be modulated with a frequency that is related to the shivering frequency.

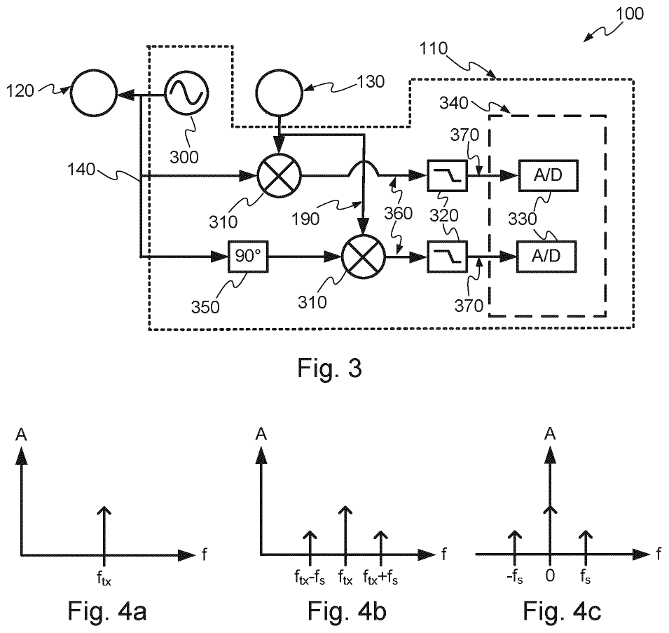

In FIG. 3, a schematic view of the shivering detection apparatus 100 is shown and based on this and the theoretic explanation given above, a detailed technical explanation of the shivering detection apparatus 100 will be given. The shivering detection apparatus 100 comprises, as seen in FIG. 1, a transmit unit 120, a receive unit and a controlling unit 110. In FIG. 3, the controlling unit 110 is detailed with a dotted line, this is to emphasize that it is an optional and further this, not all parts or functions comprised in the dotted area of FIG. 3 need be comprised in, or performed by the controlling unit 110. Some of these functions, as will be explained later, may be accomplished using simple passive or semiconductor components. With that said, the controlling unit 110 may comprise a signal source 300, at least one multiplier 310, at least one low pass filter 320 and at least one analogue to digital converter 330, from here forth AD-converter 330. The AD-converter(s) may be part of a digital signal processing unit 340 of sorts. If more than one multiplier is comprised in the shivering detection apparatus 100, the apparatus 100 may further comprise at least one phase shifting unit 350.

With continued reference to FIG. 3, the signal source 300 is arranged to provide the reference signal 140 of the transmit frequency fₜₓ to the transmit unit and the at least one multiplier 310. The receive unit 130 is operatively connected to at least one multiplier 310. Preferably, two multipliers 310 are provided and both are operatively connected to the transmit unit 120 and the receive unit 130. As shown in FIG. 3, one multiplier 310 is operatively connected to the signal source 300 via the phase shifting unit 350. The phase shifting unit 350 is in FIG. 3 shown as a 90 degree phase shift but the skilled person realizes that this may very well be realized in numerous different ways and that the important feature is that the signals coming out from the multipliers 310 should be orthogonal to one another. This setup with two multipliers 310 as shown in FIG. 3 depicts e.g. a quadrature multiplier, quadrature mixer or quadrature phase detector. The output of the multiplier(s) 310 is a down converted receive signal 360 which is provided to the low pass filter(s) 320 to provide a receive channel 370. The low pass filer(s) 320 may be provided with a DC-blocking function making them efficiently band pass filter(s) but for the teachings of this disclosure the type of filter will be correctly chosen by the skilled person and the naming thereof is of minor importance. However, it should be noted that due to ingenious design of the shivering detection apparatus 100, the filters may be implemented by a simple series resistor and shut capacitor making it a very cost effective and simple filter. As will be explained in further detail below, the receive channel 370 will oscillate with a corresponding frequency as the reflecting surface 150. There are many ways to identify the shivering frequency fₛ in the receive channel 370 and in FIG. 3 but one is shown. The receive channel 370 is, in FIG. 3, converted into digital signals by the AD-converter(s) 330. These digital signals are added together to form a complex sum which is subjected to a Fast Fourier Transform, FFT from here after, providing a representation of the shivering, if any, in the frequency domain.

The conversion into digital signal by the AD-converter(s) 330 may be replaced by a comparator circuit in order further reduce the cost of the already cost-effective shivering detection apparatus 100. As will be apparent from later explanations, the down converted receive signal 360 will vary in amplitude with the shivering frequency fₛ but with a DC offset that is depending on the distance to the shivering surface 150. By including a DC-block in the filter 370 a peak detector comprising a series diode follower by a shunt capacitor may be implemented to provide a DC-voltage level relative to the amplitude of the shivering. This DC-voltage may be compared to a reference voltage in order to detect shivering.

In FIGS. 4a-c, simplified graphs of the frequency content of signals at different places of the shivering detection apparatus 100 is shown with frequency f on the horizontal axis and amplitude A on the vertical axis. In FIG. 4a, the frequency content of the transmit signal 170 is shown. The transmit signal 170 is, in this example, shown as single frequency signal of the transmit frequency fₜₓ. In FIG. 4b, the reflected signal 180 is shown which is the transmit signal 170 after it has been reflected by a reflecting surface 150 shivering with shivering frequency fₛ. From the theories presented above, it is clear that the transmit frequency fₜₓ is modulated by the shivering frequency fₛ providing two sidebands, one at the carrier frequency minus the modulation frequency, fₜₓ−fₛ in FIG. 4b, and one at the carrier frequency plus the modulation frequency, fₜₓ+fₛ in FIG. 4b. The frequency content shown in FIG. 4b is what the frequency content of the receive signal will look like, i.e. the reflected signal 180 after it has been received by the receiving unit 130. As seen in FIG. 3, the receive signal 190 is multiplied with a signal of the transmit frequency fₜₓ to provide the down converted receive signal 360. The frequency content of the down converted receive signal 360 is shown in FIG. 4c. Note that FIG. 4c only shows the, for the conceptual explanation, relevant parts of the mixing products of the down converted receive signal 360. From FIG. 4c, is can be seen that the multipliers 310 have shifted the spectrum shown in FIG. 4b into a frequency spectrum centered around 0 Hz. The down converted receive signal 360 is what is subjected to the low pass filter(s) which may, as mentioned earlier, comprise a DC-blocking component to remove the part of the spectrum at 0 Hz leaving only the shivering frequency fₛ in the frequency spectrum.

Based on the above, the receive channel 370 is a signal of the shivering frequency fₛ. This means that if the reflecting surface 150 is shivering, the receive channel 370 will reflect this by having similar time variant behavior as the reflecting surface 150. Earlier, with reference to FIG. 3, the shivering was detected in the receive channel 370 by digitalizing the receive channel 370 and analyzing the frequency content of the receive channel 370 by FFT. This is beneficial since it allows a complex analysis to be performed and a graphical presentation of the shivering can be presented to a user of the system. Complex digital decision criteria may be implemented and e.g. any cooling of a patient may be accurately controlled from the analysis of the FFT. However, a much simpler, but rougher, detection may be performed simply by having the receive channel 370 directly provided to a comparator comparing the receive channel 370 to a shivering threshold level. This shivering threshold may be a configurable or predefined level. Once the receive channel 370 is above the shivering threshold level, a signal may be provided that signals shivering of the reflecting surface 150. In order to successfully implement a comparator solution, the slew rate of the low pass filter(s) 320 needs to be adjusted and the DC-components needs to be removed from the down converted receive signal 360. Once realized, the implementation of the comparator solution, including the design of the low pass filter(s) 320, is known to the skilled person.

Returning to the physical modulation occurring in reflected signal 180 due to shivering or vibration of the reflecting surface 150 an alternative description will be given. The distance d between the transmit unit 120 and the reflecting surface 150 can, when the reflecting surface 150 is vibrating, be described according to Eqn. 4 below. In Eqn. 4, the average distance d₀ between the reflecting surface and the receive 130 and/or transmit unit 120 is a function of the amplitude of shivering ds changed similarly to the velocity v in Eqn. 2 above.

d(t)=d₀+dₛ cos(2πfₛt+θₛ) Eqn. 4

When measuring the distance d many different types of wireless distance measurement equipment are used. Typically these devices measures the distance by measuring the time from transmission of the transmit signal 170 to the reception of the receive signal 180, these systems are often referred to as time of flight systems. Versions are used where the transmit signal 170 is formed as a chirp in order to ensure e.g. phase alignment etc.

What the inventor behind this disclosure as realized is that if the transmit signal 170 is continuously compared to the reflected signal 180, the average distance d₀ between the reflecting surface 150 and the transmit and/or receive unit 120, 130 nothing more than a complex phase shift. One way of describing the theory of operation is to assume a four quadrant overlap phase detector. The phase detector splits the receive signal 190 into two channels where one is multiplied with a signal that is in phase with the transmit signal 170, this channel is called the I-channel (in-phase). The other channel is multiplied with a signal that is 90 degrees out of phase with the transmit signal 170, this channel is called the Q-channel (quadrature phase). After multiplication and filtering out high frequency variations, the I-channel comprises a time variant signal S_{I} according to Eqn 5a, and the Q-channel comprises a time variant signal S_{Q} according to Eqn. 5b.

S_{I}(t)=1/2 cos(∅₀)−1/2δₛ cos(2πfₛt+θₛ)sin(∅₀) Eqn. 5a

S_{Q}(t)=1/2 sin(∅₀)+1/2δₛ cos(2πfₛt+θₛ)cos(∅₀) Eqn. 5b

Where ∅₀ and δₛ are described in Eqns. 6a and 6b below:

$${∅}_{0} = 2 π \frac{{f}_{tx}}{{v}_{0}} 2 {d}_{0} \qquad Eqn . 6 a$$

$${δ}_{s} = 2 π \frac{{f}_{t x}}{{v}_{0}} 2 {d}_{s} \qquad Eqn . 6 b$$

These time variant signals S_{I}, S_{Q} are digitalized and are added together providing a complex sum S, according to Eqn. 7:

S(t)=2S_{I}(t)+j2S_{Q}(t) Eqn. 7

Which, through, e.g. Eulers equations may be rewritten as shown in Eqn. 8:

S(t)=e^{i∅}^{₀}(1+jδₛ cos(2πfₛt+θₛ)) Eqn. 8

From Eqn. 8, the effect of the average distance d₀ (comprised in ∅₀ in Eqn. 8) between the transmit and/or receive unit 120,130 is a complex phase shift and the shivering has been isolated from it.

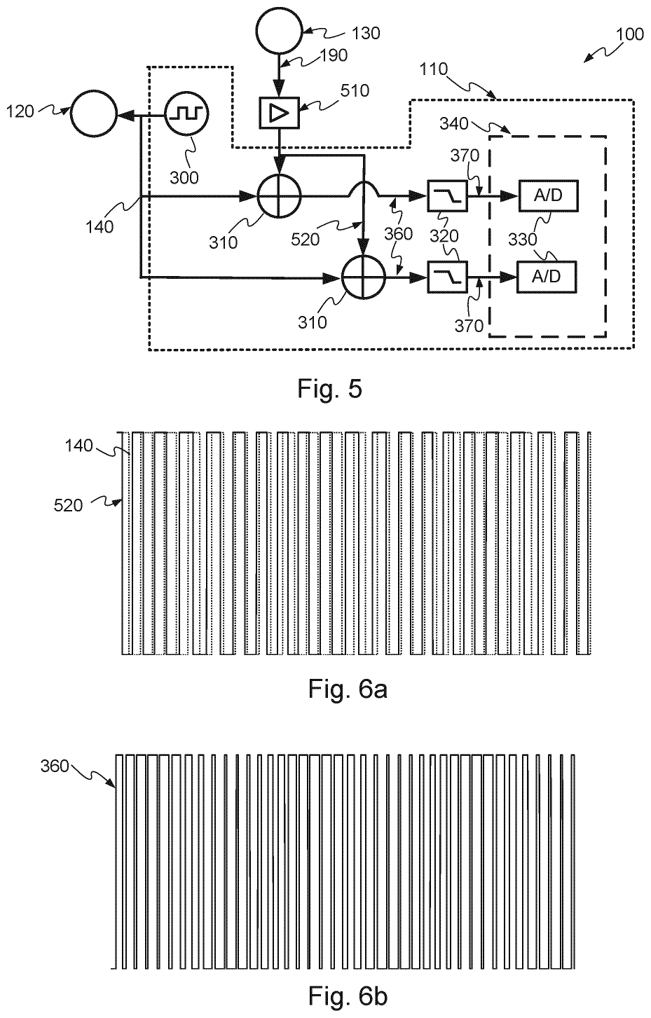

The teachings above enables the shivering detection apparatus 100 to be even further simplified and implemented using cheap standard electrical components. A schematic diagram of such a solution is shown in FIG. 5. The signal source 300 is implemented using a square wave generator providing the reference signal 140 as a square wave of the transmit frequency fₜₓ. The rising edge of the reference signal 140 may be used to trigger the transmit unit 120 and provide the in phase reference to one multiplier 310. In FIG. 5, the multipliers are shown as adders 310. The falling edge of the reference signal 140 may be used to provide the quadrature phase reference to another adder 310. In addition to what is shown in FIG. 3, in FIG. 4, the receive signal 190 is subjected to a high pass filter 510 before it is provided to the multiplier(s) 310. The high pass filter 510 is adapted to allow substantially all power at the transmit frequency fₜₓ and the modulation frequencies, fₜₓ+fₛ and fₜₓ−fₛ, to pass through them. The high pass filter 510 provides a filtered receive signal 520 to the multiplier(s) 310. The high pass filter 510 may be provided with an amplifying function such that the filtered receive signal 520 is amplified to a maximum amplitude that is substantially the same as the maximum amplitude of the signal provided by the signal source 510. Further to this, the high pass filter 510 may be provided with comparator functionality such that the filtered receive signal 520 is a square wave. Although more than one function has been associated with the high pass filter 510, it should be mentioned that the high pass filters 510 should not necessarily be considered one component or function. Rather, the high pass filter 510 may very well be several different devices or component arranged to provide the functions listed above. When the filtered receive signal 520 is a squared up signal, the multipliers 310 may be implemented using explicit OR-gates, XOR gates from now on. The XOR gates 310 will provide a positive output only when one, and only one, of the inputs is positive, they can be regarded as 1-bit adders. Note that the function of the system is the same if e.g. XNOR gates where to be used in place of the XOR gates 310. The outputs from the XOR gates 310 is subjected to the low pass filter(s) 320 with a cut-off frequency adapted to that of the shivering to be detected. Typically, the cut-off frequency is about 500 Hz. These signals are digitalized by the AD-converters 330 allowing the calculation of the complex sum as per Eqn. 7 above.

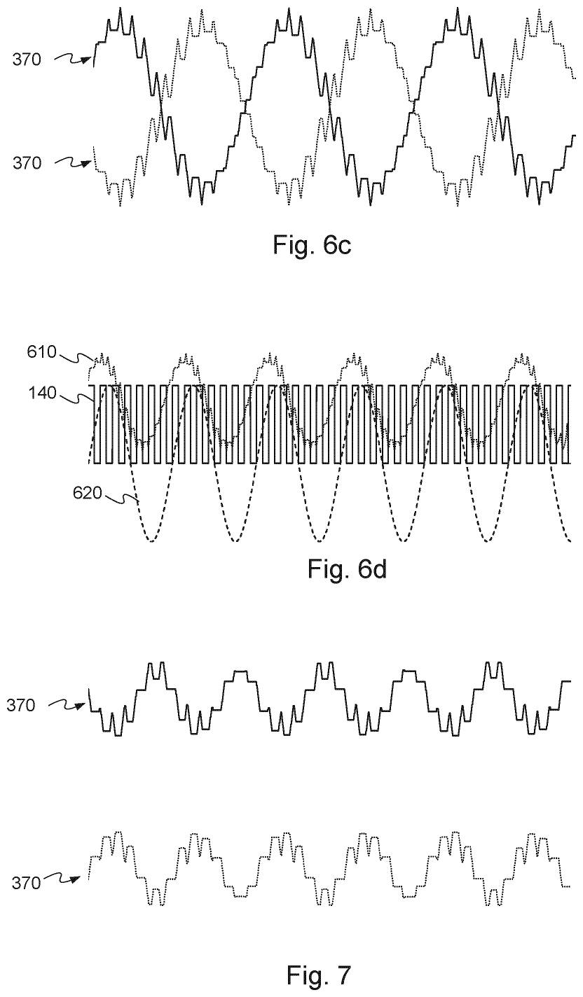

In order to further the explanation of the embodiment detailed with reference to FIG. 5 some simplified signal graphs are shown if FIGS. 6a-d depicting signals at different locations of the shivering detection apparatus 100. The vibration that will be seen in the graphs is of a shivering frequency fₛ of one 7^{th} of the transmit frequency fₜₓ. This is too small a difference for efficient filterering and is used for illustrative purposes, typically there would be a factor 50-200 between the shivering frequency fₛ and the transmit frequency fₜₓ. The transmit signal 170 is, in all the FIGS. 6a-d, modulated y the shivering of the reflecting surface 150 located at a distance of (2n+1)*90° from the receiving unit 160 and the transmit unit 120 where n is any positive integer number. In other words, the receive signal 190 is 90° out of phase compared to the reference signal 140. In FIG. 6a, the reference signal 140, dotted line, is shown together with the filtered receive signal 520 squared up, solid line. The filtered receive signal 520 is modulated by the shivering as detailed above and this can be seen as the two signals 140, 520 in FIG. 6a being of different frequencies. FIG. 6b presents the down converted receive signal 360 as a result of the one-bit addition by the XOR gate 310, or one bit multiplication by an XNOR gate, of the filtered receive signal 520 and the reference signal 140 as shown in FIG. 6a, i.e. the in phase signal. FIG. 6c illustrates the receive channels 370 with the in phase signal as a solid line and the quadrature phase signal as a dotted line. As seen from FIG. 6c, the sum of the receive channels 370 will be constant. These receive channels 370 are the signals that are sampled into digital signals and combined into a complex sum providing a vector representation of the shivering with an amplitude that changes with the shivering frequency fₛ and also a phase that changes with the shivering frequency fₛ. In FIG. 6d the phase signal 610 of the shivering vector is shown as a dotted line together with, for reference, the sinusoidal shivering signal 620 as a dashed line and the reference signal 140 as a solid line. For the sake of completeness, the shivering vector amplitude is calculated as the square root of the sum of each of the two receive channels 370 squared. The phase of the shivering vector is the angle of the shivering vector as the hypotenuse in a right triangle with the receive channels 370 making up the catheti.

When the distance between the shivering surface 150 and the transmit and receive devices 120, 130 changes away from a distance corresponding to a 90° phase between the receive signal 190 and the reference signal, the amplitude swing of the receive channels 370 will decrease. In FIG. 7, the receive channels 370 can be seen when the distance between the shivering surface 150 and the transmit and receive devices 120, 130 corresponding to a phase shift of 180° between the receive signal 190 and the reference signal. Assuming that he noise of the receive signal 190 is the same in FIG. 6c and FIG. 7, typically this would be the thermal noise when comparing FIG. 7 to FIG. 6c, it is evident that a Signal to Noise Ratio, SNR, of the receive channels 370 will be much lower when the distance approaches a 180° phase shift.

The conclusions above have made the inventor realize that although the absolute distance between the shivering surface 150 and the receive and transmit device d₀ is of little concern, it should be kept as close to a 90° phase shift as possible in order to maximize the sensitivity of the system.

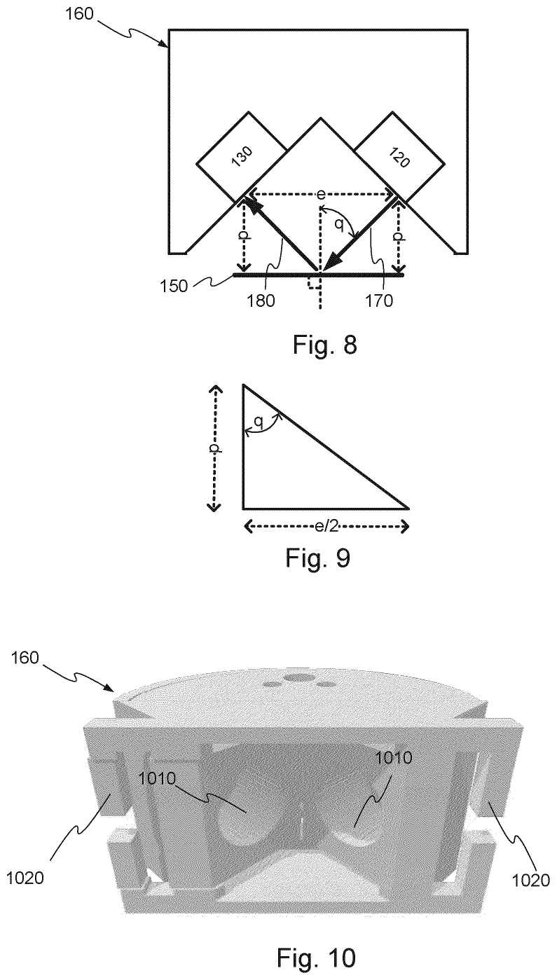

FIG. 8 shows a schematic view of the sensor head 160 presented with reference to FIG. 1 earlier. The sensor head 160 is provided to ensure a good alignment between the transmit unit 120, the receive unit 130 and the reflecting surface 150. The sensor head comprises the transmit unit 120 arranged such that the transmit signal 170 is reflected by the reflecting surface 150 at an incident angle q. The sensor head further comprises the receive unit 130 which is arranged to receive at least a portion of the reflected signal 180. The sensor head 160 is configured such that it can be placed on the reflecting surface 150 and the incident angle q will be a function of the distance d between the transmit unit 120 and the receive unit 130 and the reflecting surface and also the distance e between the transmit unit 120 and the receive unit 130. This is illustrated in FIG. 9.

The incident angle q has to be chosen carefully, a too large incident angle q will risk the receive unit 130 receiving a portion of the transmit signal 170 without it having been reflected by the reflecting surface 150. If the incident angle q is too low, a portion of the reflected signal 180 will reflect back to the transmit unit 120 and energy would be wasted. This is mainly due to the beam width of the transmit unit 120 and will depend on choice of transmit unit and transmission frequency fₜₓ. Typically, higher transmission frequency will allow for smaller incident angle q and also smaller physical devices.

Note that the distance d, will be substantially constant when the sensor head 160 is arranged on a e.g. a patient undergoing therapeutic hypothermia since any vibration or movement of the patient will move the sensor head 160 equally to the vibration or movement of the patient. The shivering measured by the sensor head 160 are typically shivering of a micro shivering type and these are detectable regardless if the sensor head 160 moves with the patient. This is an effect of micro shivering having low amplitude and vary across the body of the patient. Based on the knowledge presented in the previous sections, it should be noted that the distance d should be chosen, based on transmit frequency fₜₓ and speed of the transmit signal 170, such that the receive signal 190 is as far from a 180° degree phase shift when coherently compared to the reference signal 140. Also, the skilled person will be aware of that the area used to reflect the transmit signal 170 will have to be chosen in relation to the wavelength of the transmit signal 170.

In FIGS. 8 and 9, the sensor head 160 and its associated wave propagation has been shown in one plane. The transmit signal 170 and the reflected signal 180 will of course propagate in three-dimensional space. This result in the transmit signal 170 risk not only reflecting of the reflecting surface, but also of an interior of the sensor head. In order to reduce the risk of these unwanted reflections, the inventors have realized that inner surfaces of the sensor head 160 may be fitted with a lining material having frequency absorbent properties, i.e. reflection reducing properties. This lining material is chosen depending on the type of transmit unit 120 chosen and also depending on the transmit frequency fₜₓ. If, for instance, the transmit unit 120 is an ultrasonic transmit unit 120 for transmitting an ultrasonic acoustic signal and the transmit frequency fₜₓ is at 40 kHz, a lining material from cotton cellulose has good absorbent features.

FIG. 10 is a three dimensional view of one embodiment of the sensor head 160. The sensor head 160 is provided with at least two slots 1010 arranged to the transmit unit 120 and the receive unit 120 respectively. Further to this, the sensor head 160 comprises two mounting tabs 1020 arranged to receive means for arranging the sensor head 160 onto the body of a patient. The mounting tabs 1020 are suitable to receive e.g. an elastic strap or band that can be used to attach the sensor head 160 around the head, neck, arms or legs of the patient. In such an arrangement, the sensor head 160 can be placed at a location where the shivering is most likely to occur, typically near the area undergoing therapeutic hypothermia.

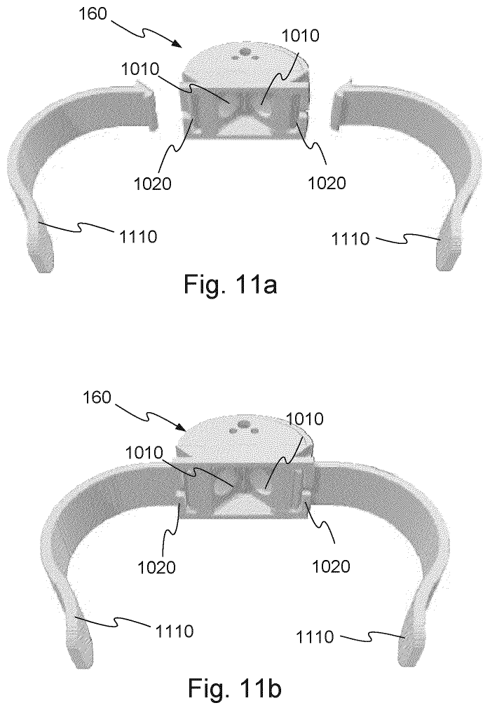

In FIGS. 11a-b, the sensor head 160 is shown together with two removable collar clips 1110 configured to removably lock into the mounting tabs 1020 as seen in FIG. 11b. The removable collar clips 1110 allows the sensor head 160 to be fastened quickly and effortlessly to e.g. the neck of the patient and the multipurpose holders enables the same sensor head 160 to be used with different fastening means, e.g. the elastic band or strap and the removable collar clips 1110. The mounting tabs 1020 allows for a reduced number of sales items and consequently reduced storage space and logistic costs. Also, the flexibility in fastening means makes the sensor head 160 easier to use in diverse situations.

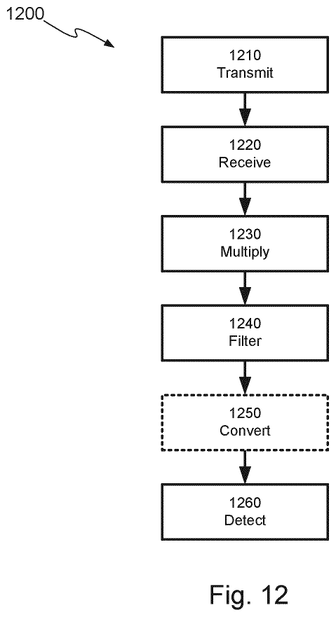

The teaching disclosed in the previous sections will now be form a shivering detection method 1200 as schematically depicted in FIG. 12. The method 1200 comprises the step of wirelessly transmitting 1210 a transmit signal 170 towards a patient. The transmitting 1210 may typically be performed as described when detailing the transmit unit 120 in the shivering detection apparatus 100.

The method 1200 further comprises receiving 1220, a reflected signal 180 as a portion of the transmit signal 170 reflected by the patient. The wireless reflected signal 130 is received 1220 and provided as the wired receive signal 190. The receiving 1220 may typically be performed as described when detailing the receive unit 120 in the shivering detection apparatus 100.

Additionally, the method 1200 comprises the step of multiplying 1230, the receive signal 190 with a reference signal 140 to provide one or more down converted receive signals 360 centered at 0 Hz. The multiplying 1230 may be performed in any way as disclosed in precious sections, e.g. using mixers 310, XOR-gates 310 or XNOR-gates 310.

The method 1200 further comprises filtering 1240 the down converted receive signals 360 to provide one or more receive channels 370. The filtering 1240 may be performed in any suitable way as described in the previous sections.

Further to this, the method 1200 may comprise converting 1250 the receive channels 370 to digital signals by means of e.g. an analogue to digital converter 330.

The method 1200 comprises detecting 1260 the shivering of the patient based on the one or more digital receive channels by means of e.g. a comparator or processing unit 340. As taught in the previous sections, any shivering will be detectable in the digital receive channels.

In the previous sections, an apparatus 100 with an associated method 1200 and sensor head 160 has been presented. The teachings are of a novel an extremely cost effective way of detecting shivering of a patient. The use of coherent detection and acoustic waves allows the solution to be implemented using off the shelf components and the total cost is much lower than that of competing system. The form factor is extremely small making the apparatus 100 portable and mountable by medical staff or emergency personnel on a patient without the apparatus obstructing examination or treatment of the patient. Further to this, the device may function as a stand alone device in detecting shivering of a patient, or it may be integrated into other medical equipment as a sensor to e.g. control cooling of patient etc.

1. A shivering detection apparatus for detecting shivering of a patient, the apparatus comprising:
a control unit operatively connected to
a transmit unit for transmitting a transmit signal based on a reference signal of a transmit frequency (fₜₓ) towards the patient, and
a receive unit for receiving a receive signal as a portion of a reflected signal that includes a portion of the transmit signal reflected by the patient;
wherein the control unit is configured to compare the receive signal to the reference signal and to detect shivering of the patient as differences between the receive signal and the reference signal.

2. The apparatus of claim 1, wherein the control unit comprises:
a multiplying unit for multiplying the receive signal with the reference signal to provide one or more down converted receive signals centered at 0 Hz;
a filtering unit for low pass filtering the down converted receive signals to provide one or more receive channels;
a converting unit for converting the one or more receive channels to provide one or more digital receive channels; and
a processing unit for detecting shivering of the patient based on the one or more digital receive channels.

3. The apparatus of claim 2, wherein the multiplying unit comprises:
a first multiplier arranged to multiply the receive signal with an in phase reference of the reference signal to provide an in phase receive channel; and
a second multiplier arranged to multiply the receive signal with a quadrature phase reference of the reference signal to provide a quadrature phase receive channel;
wherein the quadrature phase reference is of an orthogonal phase to the in phase reference.

4. The apparatus of claim 3, wherein the converting unit comprises:
a first analogue to digital converter arranged to convert the in phase receive channel to provide a digital in phase receive channel; and
a second analogue to digital converter arranged to convert the quadrature phase receive channel to provide a digital quadrature phase receive channel.

5. The apparatus of claim 4, wherein the processing unit is configured to:
add the digital in phase receive channel and the digital quadrature phase receive channel to provide a complex sum; and
subject the complex sum to a Fast Fourier Transform, FFT, to provide a frequency representation of the complex sum.

6. The apparatus of claim 5, wherein shivering is identified if the frequency representation of the complex sum comprises at least one frequency component above a shivering threshold.

7. The apparatus of claim 1, further comprising providing a shivering control signal for controlling apparatuses external to the shivering detection apparatus and/or for alerting personnel to the presence of shivering.

8. The apparatus of claim 1, wherein the transmit unit is an ultrasonic transducer for transmitting an ultrasonic acoustic signal and the receive unit is an ultrasonic receiver for receiving an ultrasonic acoustic signal.

9. The apparatus of claim 3, wherein the one or more multipliers is a logical exclusive-or, XOR, gate.

10. The apparatus of claim 2, wherein the receive signal and the reference signal are normalized to a common level prior being provided to the multiplying unit.

11. The apparatus of claim 10, wherein the normalizing is realized by one or more analogue comparators.

12. The apparatus of claim 1, wherein the patient is subjected to therapeutic hypothermia.

13. A shivering detection method for detecting shivering of a patient, the method comprising:
transmitting, wirelessly by a transmit unit, transmit signal of a transmit frequency (fₜₓ) based on a reference signal towards the patient;
receiving, by a receive unit, a reflected signal as a portion of the transmit signal reflected by the patient, to provide a receive signal;
multiplying, by one or more multipliers, the receive signal with the reference signal to provide one or more down converted receive signals centered at 0 Hz;
filtering, by a low pass filter, the one or more down converted receive signals to provide one or more receive channels;
converting, by at least one analogue to digital converter, the one or more receive channels to provide one or more digital receive channels;
delecting, by a processing unit, shivering of the patient based on the one or more digital receive channels.

14. The method of claim 13, wherein the step of multiplying further comprises:
multiplying, by a first multiplier, the receive signal with an in phase reference of the reference signal to provide an in phase receive channel; and
multiplying, by a second multiplier, the receive signal with a quadrature phase reference of the reference signal to provide a quadrature phase receive channel;
wherein the quadrature phase reference is of an orthogonal phase to the in phase reference.

15. The method of claim 14, wherein the step of converting further comprises:
converting, by a first analogue to digital converter, the in phase receive channel to provide a digital in phase receive channel; and
converting, by a second analogue to digital converter, the quadrature phase receive channel to provide a digital quadrature phase receive channel.

16. The method of claim 15, wherein the step of detecting further comprises, by the processing unit;
adding the digital in phase receive channel and the digital quadrature phase receive channel to provide a complex sum; and
subjecting the complex sum to a Fast Fourier Transform, FFT, to provide a frequency representation of the complex sum.

17. A sensor head for use with the shivering detection apparatus of claim 1 to detect shivering of a patient, the sensor head comprising:
the transmit unit and the receive unit of the shivering detection apparatus;
wherein the transmit unit and the receive unit are arranged in the sensor head, such that the transmit signal of the transmit frequency (fₜₓ) transmitted by the transmit unit is reflected by the patient at an incident angle (q) forming the reflected signal, and such that at least a portion of the reflected signal is detectable by the receive unit.

18. The sensor head of claim 17, wherein the incident angle (q) is less than 55 degrees.

19. The sensor head of claim 17, wherein at least one inner surface of the sensor head is provided with a lining material that comprises absorbing features of signals of the transmit frequency (fₜₓ).

20. The sensor head of claim 19, wherein the transmit signal is an ultrasonic acoustic signal;
wherein the transmit frequency (fₜₓ) is 40 Hz; and
wherein the lining material is cotton cellulose cloth.

21. The sensor head of claim 17, wherein the sensor head further comprises at least two mounting tabs arranged to receive an elastic band and/or a removable collar clip.

22. The sensor head of claim 21, wherein the sensor head is configured to be arranged on the temple of the patient by means of the clastic band and/or on the neck of the patient by means of the removable collar clips.
